(19) United States
(12) Patent Application Publication
(10) Pub. No.: US 2024/0277253 A1
(43) Pub. Date: Aug. 22, 2024

(54) COUGH DETECTION SYSTEM, METHOD, AND DEVICE

(71) Applicant: Respiri Limited, Melbourne, Victoria (AU)

(72) Inventors: Ronald Scott Platt, Melbourne (AU); Samaneh Sarraf Shirazi, Melbourne (AU); Marjan Mikel, Melbourne (AU)

(21) Appl. No.: 18/612,890

(22) Filed: Mar. 21, 2024

Related U.S. Application Data
(63) Continuation-in-part of application No. PCT/AU2022/051147, filed on Sep. 23, 2022.

(30) Foreign Application Priority Data
Sep. 22, 2021 (AU) 2021903056

(51) Int. Cl. A61B 5/08; A61B 5/00; A61B 5/01; A61B 5/113; A61B 7/00

(52) U.S. Cl. CPC A61B 5/0823; A61B 5/01; A61B 5/1135; A61B 5/6833; A61B 5/7203; A61B 5/7289; A61B 7/003

(57) ABSTRACT

A wearable apparatus for a patient for use in cough detection, comprising: a movement sensor configured to generate measurements of a movement of the patient, the movement sensor configured to be worn by the patient; a sound sensor configured to generate measurements of breathing sounds of the patient; a controller interfaced with the movement sensor and the sound sensor; and a housing for locating the movement sensor, sound sensor, and controller, wherein the controller is configured to: obtain a movement signal corresponding to the measurements of the movement of the patient made by the movement sensor; obtain a sound signal corresponding to the measurements of the breathing sounds of the patient made by the sounds sensor; generate thoracic movement data from the movement signal; generate breathing data from the sound signal; and generate respiratory data comprising the thoracic movement data and the breathing data, and further comprising correlation information configured to enable identification of contemporaneous breathing events within the thoracic movement data and the breathing data; provide correlated respiratory data to a processing module configured for identifying specific breathing events at least including coughing events, such that a coughing event is identifiable from the correlated respiratory data according to a coincidence of identification of a predefined sound pattern within the breathing data and identification of a predefined movement pattern within the thoracic movement data.

### FIELD OF THE INVENTION

The present invention generally relates to a device, method, and system for monitoring breathing of a patient and identifying instances of coughing.

### BACKGROUND TO THE INVENTION

Inflammatory diseases of the airways of the lungs can cause variable and reoccurring symptoms of wheezing, coughing and shortness of breath. A conventional device used to monitor for such symptoms is a peak flow meter; a handheld device which measures a person's maximum speed of expiration and thus the degree of obstruction in their airways. The effectiveness of a peak flow monitor for self-diagnosis of symptoms is, however, limited, due to the wide range of ‘normal’ values of peak flow and the high degree of variability in results.

Wheeze monitors, such as the Airsonea® by iSonea®, have been developed which monitor vibrations of the trachea using a contact sensor placed on the skin in proximity to the trachea. Such devices use piezoelectric sensors to pick up breath sounds which can be analysed to determine whether the patient is wheezing and to what degree.

Wheezo”®, by the present Applicant, comprises a hand-held recording device and associated software. The system is used by patients for long term management of their asthma. Patients use the recording device to make short, typically 30 second, recordings of their breathing sounds by placing the recording device on their trachea. The device communicates via Bluetooth with a smartphone running an application we have developed that transmits the recording to a centralized database infrastructure for analysis and future review. Software within the centralized database infrastructure is used to develop relationships between the sound recordings, medication usage and extraneous factors such as weather, air quality, pollen count, etc. These relationships provide information for patients to better understand what practices worsen or improve their baseline asthmatic condition. This information is provided to the patients via the smartphone application.

A component of the software infrastructure is an algorithm to analyze the breath sound recordings and detect “wheeze” which results from restriction of large and medium airways in the lungs. Wheeze is a key indicator of asthma so regular measurement of wheeze will reveal long-term changes in asthma. Physicians use a stethoscope placed on the chest or back to listen for wheeze sounds in a patient. Wheeze is heard in breathing sound as a whistling or singing like pitch. The analysis algorithm processes the sound signal and marks periods containing pitch that is characteristic of wheeze.

The recording device also has a second microphone for simultaneously recording ambient sound when measuring breathing. The recordings from the second microphone are used in order to distinguish between true wheeze and background noise. In particular, this second microphone enables periods with excessive extraneous background noise to be eliminated from analysis.

It is desired to provide further developments in respect of devices for monitoring breathing.

### SUMMARY OF THE INVENTION

According to an aspect of the present invention, there is provided a wearable apparatus for a patient for use in cough detection, comprising: a movement sensor configured to generate measurements of a movement of the patient, the movement sensor configured to be worn by the patient; a sound sensor configured to generate measurements of breathing sounds of the patient; a controller interfaced with the movement sensor and the sound sensor; and a housing for locating the movement sensor, sound sensor, and controller, wherein the controller is configured to: obtain a movement signal corresponding to the measurements of the movement of the patient made by the movement sensor; obtain a sound signal corresponding to the measurements of the breathing sounds of the patient made by the sounds sensor; generate thoracic movement data from the movement signal; generate breathing data from the sound signal; and generate respiratory data comprising the thoracic movement data and the breathing data, and further comprising correlation information configured to enable identification of contemporaneous breathing events within the thoracic movement data and the breathing data; provide correlated respiratory data to a processing module configured for identifying specific breathing events at least including coughing events, such that a coughing event is identifiable from the correlated respiratory data according to a coincidence of identification of a predefined sound pattern within the breathing data and identification of a predefined movement pattern within the thoracic movement data.

Optionally, the housing comprises an external surface defining an interface portion substantially comprising a first face of the body, wherein, in-use, the interface portion is configured to be proximal a surface of the patient. The interface portion may comprise at least one protrusion configured for contact with the patient, the, or each, protrusion may be associated with a sensor of the wearable apparatus.

The wearable apparatus optionally further comprises a battery and/or a port for connecting an external power supply.

The wearable apparatus optionally further comprises a communication module interfaced with the controller, the communication module enabling data communication with a processing server via at least one wired and/or at least one wireless protocol.

The wearable apparatus optionally further comprises a holder, the holder having a shape complementary to a profile of at least the interface portion of the housing such that the housing is attachable to and detachable from the holder, wherein the holder is configured to securely hold the housing during use. The holder may be configured to be worn by the patient such that, in-use, the housing may be worn by the patient via the holder. An aperture may be provided in the holder for the, or each, protrusion of the interface portion, such that the, or each, protrusion extends through its associated aperture thereby enabling contact between the protrusion and a surface of the patient. The holder and the housing may comprise complementary locating means thereby ensuring a desired relative positioning, in-use, of the housing on the holder. The holder may comprise attachment means for enabling secure attachment of the holder and the housing. The attachment means may include one or more clip portions configured to interact with associated complementarily shaped one or more end portions of the housing. The housing may include a recess comprising an operational switch and the holder may comprise a complementary power activating being a protrusion configured to actuate the operational switch when the housing is securely held by the holder.

The wearable apparatus optionally further comprises securing means configured for attaching to the holder to enable the holder to be secured to the patient. The securing means may comprise an adhesive element having a geometry such that, when attached to the holder, one or more tabs extend away from the holder, said one or more tabs may include an adhesive layer for securing the, or each, tab to the patient.

The wearable apparatus optionally further comprises an auxiliary microphone arranged to substantially measure background sound to enable removal of said measured background sound from the measurements of the breathing sounds of the patient.

The wearable apparatus optionally further comprises a temperature sensor configured to produce temperature measurements of a temperature of the patient, and the controller is optionally further configured to: obtain a temperature signal corresponding to the measurements of the temperature of the patient made by the temperature sensor; generate temperature data from the temperature signal; and include the temperature data with the respiratory data wherein the correlation information is further configured to enable identification of contemporaneous temperatures of the patient associated with specific breathing events.

The sound sensor may comprise a microphone such as a MEMS microphone. The movement sensor may comprise an accelerometer.

In an embodiment, the wearable apparatus comprises the processing module, such that the controller implements the processing module.

According to another aspect of the present invention, there is provided a cough detection system for the detection of coughs by a patient, the system comprising; a wearable apparatus as disclosed above; and the processing module

Optionally, the processing module is configured to: analyse the breathing data to identify instances of the breathing signal consistent with a predefined characteristic cough pattern, analyse the thoracic movement data to determine instances of a thoracic movement corresponding to a predefined characteristic movement pattern, an in response to identifying a correlation, in time, of an instance of a predefined cough pattern and an instance of a predefined movement pattern, assign the instances as corresponding to a cough. The predefined characteristic cough pattern may be characterised by a relatively large amplitude compared to non-cough breathing sounds having short duration bursts with a broad spectral pattern. The predefined characteristic cough pattern may at least in part be defined by one or more settable parameters, such that a user is enabled to configure the predefined characteristic cough pattern. The predefined characteristic movement pattern may be characterised by a relatively large movement of the patient's thorax compared to the magnitude of the amplitude of the signal generated by the sound sensor. The predefined characteristic movement pattern may at least in part be defined by one or more settable parameters, such that a user is enabled to configure the predefined characteristic movement pattern.

The processing module is optionally implemented by the controller of the wearable apparatus. Alternatively, the system further optionally comprises a processing sever separate to the wearable apparatus, the processing module being implemented by the processing server, and the wearable apparatus being configured to communicate the respiratory data to the processing server via one or more data communication protocols. The respiratory data may be stored on the wearable apparatus and communicated to the processing sever subsequently to the measurements being made of the patient. The respiratory data may be communicated to the processing sever while the measurements of the patient are being made.

According to yet another aspect of the present invention, there is provided a method for processing respiratory data, wherein the respiratory data is obtained, at least in part, by a wearable apparatus comprising a sound sensor and a movement sensor, comprising the steps of: obtaining respiratory data comprising thoracic movement data corresponding to measurements of a movement of the patient's thorax made by the movement sensor and the breathing data corresponding to measurements of breathing sounds of the patient made by the sound sensor, and further comprising correlation information configured to enable identification of contemporaneous breathing events within the thoracic movement data and the breathing data; analysing the breathing data to identify instances of the breathing signal consistent with a predefined characteristic cough pattern, analysing the thoracic movement data to determine instances of a thoracic movement corresponding to a predefined characteristic movement pattern, and in response to identifying a correlation, in time, of an instance of a predefined cough pattern and an instance of a predefined movement pattern, assigning the instances as corresponding to a cough.

The method optionally further comprises the steps of: obtaining, by a controller of the wearable apparatus, a movement signal corresponding to the measurements of the movement of the patient made by the movement sensor; obtaining, by a controller of the wearable apparatus, a sound signal corresponding to the measurements of the breathing sounds of the patient made by the sounds sensor; generating, by a controller of the wearable apparatus, thoracic movement data from the movement signal; generating, by a controller of the wearable apparatus, breathing data from the sound signal; and generating, by a controller of the wearable apparatus, the respiratory data.

The predefined characteristic cough pattern may be characterised by a relatively large amplitude compared to non-cough breathing sounds having short duration bursts with a broad spectral pattern. The predefined characteristic cough pattern may at least in part be defined by one or more settable parameters, such that a user is enabled to configure the predefined characteristic cough pattern. The predefined characteristic movement pattern may be characterised by a relatively large movement of the patient's thorax compared to the magnitude of the amplitude of the signal generated by the sound sensor. The predefined characteristic movement pattern may at least in part be defined by one or more settable parameters, such that a user is enabled to configure the predefined characteristic movement pattern.

As used herein, the word “comprise” or variations such as “comprises” or “comprising” is used in an inclusive sense, i.e. to specify the presence of the stated features but not to preclude the presence or addition of further features in various embodiments of the invention.

### BRIEF DESCRIPTION OF THE DRAWINGS

In order that the invention may be more clearly understood, embodiments will now be described, by way of example, with reference to the accompanying drawings, in which:

FIGS. 1A-1C show variations of a cough detection system, according to embodiments;

FIGS. 2A and 2B show a housing for sensors and a controller of the system, according to an embodiment, and FIG. 2C shows a holder for receiving the housing;

FIGS. 3A and 3B show an act of attaching the housing of FIGS. 2A and 2B to the holder of FIG. 2C;

FIG. 4 shows additional features of a holder, according to an embodiment;

FIGS. 5A and 5B show an adhesive element for use with the holder and housing;

FIG. 6 shows a method of operating the system; and

FIG. 7 shows example data of different breathing events including coughing, wheezing, and speaking.

### DESCRIPTION OF EMBODIMENTS

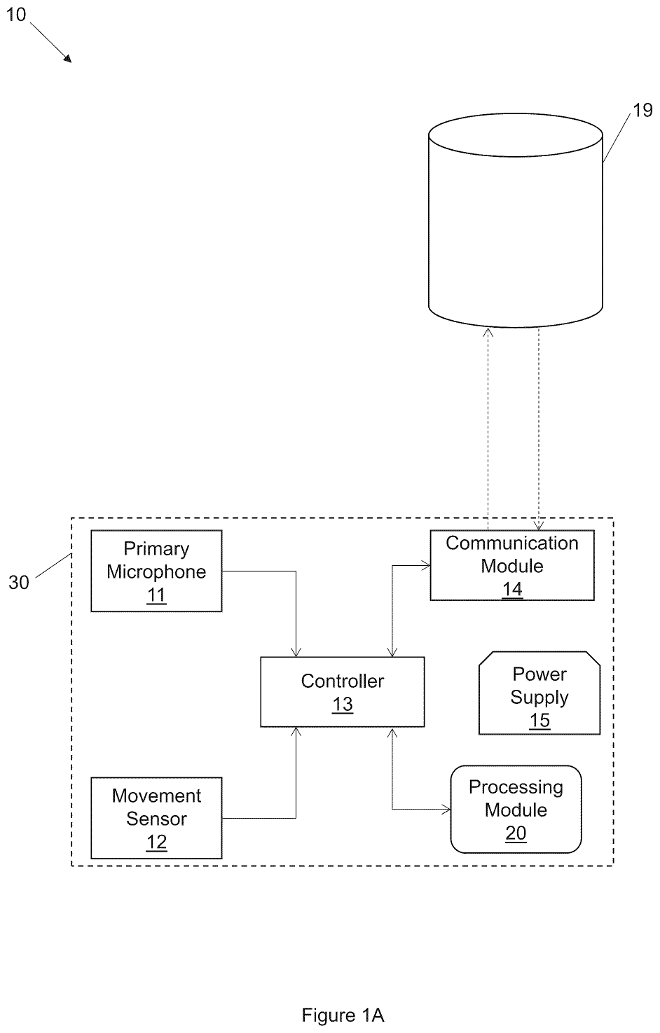

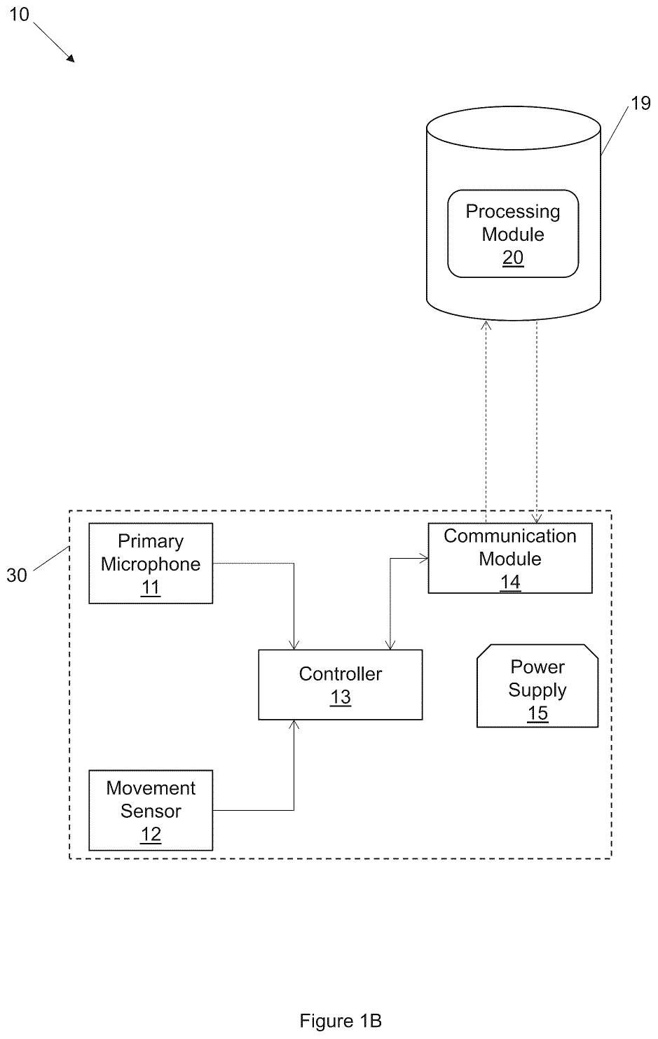

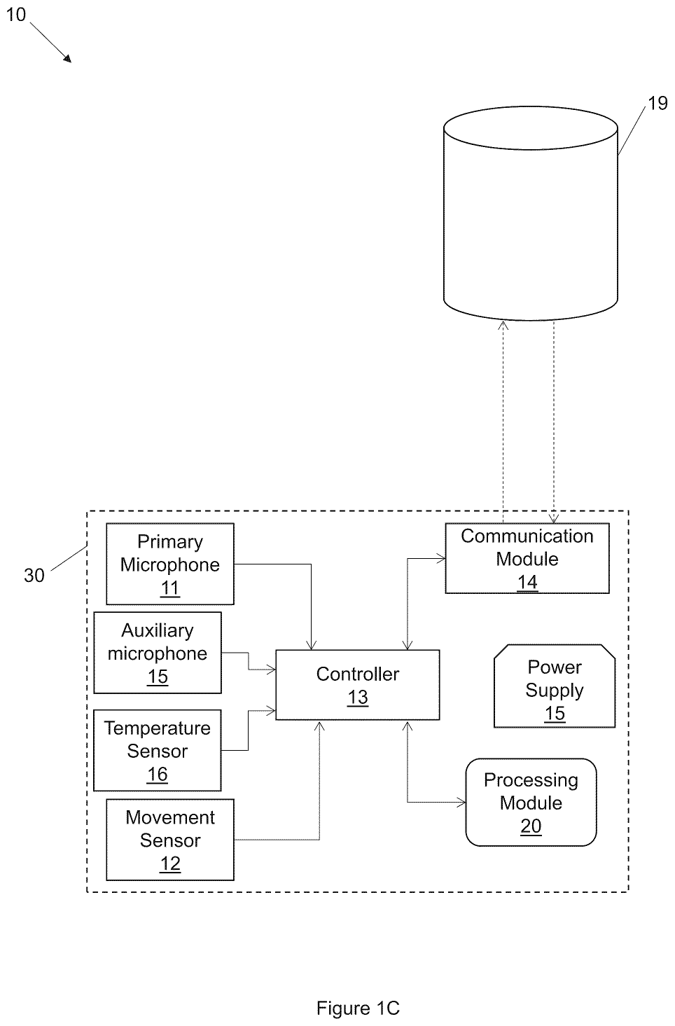

FIGS. 1A and 1B show schematic representations of features of a cough detection system 10 according to two embodiments. In each embodiment, the system 10 comprises a sound sensor 11 and a movement sensor 12, each interfaced with a controller 13. The controller 13 is also interfaced with a communication module 14 which is configured to transmit and receive data communication with a processing system 19, which is physically separate to the controller 13. The system 10 also comprises a processing module 20 for processing data obtained by the controller 12 from the sound sensor 11 and the movement sensor 12. For much of the present disclosure, it is assumed that the processing module 20 is implemented as a function of the controller 13 as shown in the embodiment of FIG. 1A. In the alternative embodiment of FIG. 1B, the processing module 20 is implemented by the processing system 19. Reference herein to the processing module 20 should be understood to correspond to either embodiment unless stated otherwise. In a variation, the functions of the processing module 20 are effectively distributed between the controller 13 and the processing system 19, such that certain functions are performed by the controller 13 and other functions are performed by the processing system 19.

In the embodiments of FIGS. 1A and 1B, the sound sensor 11, the movement sensor 12, controller 13, and communication interface 14 are associated with a housing 30 (shown as dotted lines). The processing module 20 is consequently also housed with the housing 30 with respect to the embodiment of FIG. 1A.

Also shown is a power supply 15 which can be entirely internal (for example, a battery), externally provided (e.g. via a wired cable), or both. In an implementation, the power supply 15 comprises a rechargeable battery, such as a nickel-metal hydride (NiMH), lithium-ion (Li-ion), lithium iron phosphate (LiFePO4), or lithium-ion polymer (Li-ion polymer) battery and a power supply port, for example a USB port (e.g. USB 2.x or 3.x), arranged to allow electrical charging of the rechargeable battery as well as providing a second power supply option.

The communication module 14 is configured for data communication using either a wired communication protocol or a wireless communication protocol, or both. Wired communication can be via, for example, Universal Serial Bus (USB) or a network protocol such as Ethernet. Wired communication can also comprise non-electrical communication, such as via a fibre optic cable. Wireless communication can be via, for example, WiFi (IEEE 801.11), Bluetooth, Bluetooth Low Energy, or ZigBee.

The sound sensor 11 and movement sensor 12 are arranged, in-use, to record measurements of a patient in relation to the patient's breathing. It should be understood that the term “patient” is used to illustrate a typical use case of the system 10 and is otherwise not intended to be limiting. Similarly, the term “user” is utilised to refer to health professional or other person who may be using the system 10 in a health diagnostic scenario in relation to the patient. However, the use of the term “user” should not be construed as limiting, for example, it is envisaged that a patient may utilise the system 10 for their own diagnostics, thereby effectively making the patient the same as the user. Furthermore, although health diagnostics is a primary intended use of the system 10, this should also not be considered limiting as the system 10 is anticipated to find utility in other fields.

The sound sensor 11 is configured for detecting sound vibrations, typically within one or more frequency ranges and an amplitude range consistent with various different breathing sounds that may be made by a patient. In particular, the frequency range should be sufficient for capturing breathing sounds corresponding to coughing as well as normal breathing, and typically also wheezing. The sound sensor 11 is therefore typically a suitable microphone for example, a dynamic microphone (i.e. which uses a coil of wire suspended in a magnetic field), a condenser microphone (i.e. which uses a vibrating diaphragm as a capacitor plate), or a contact microphone (i.e. which uses a crystal of piezoelectric material).

Reference is also made to the Applicant's earlier patent publication WO 2021/081589 A1 (publication date 6 May 2021), which is incorporated herein in its entirety, which describes a contact sensor for monitoring breathing of a subject. The contact sensor utilises a micro-electromechanical systems (MEMS) microphone suitably configured housing in contact with a patient to detect breathing sounds. In an embodiment, the contact sensor described in WO 2021/081589 A1 is utilised at least in relation to implementing the sound sensor 11 herein described. Other features described in this publication may be suitable for use in the system 10 herein disclosed, for example the background microphone described may be utilised as the auxiliary microphone 15 of the present disclosure.

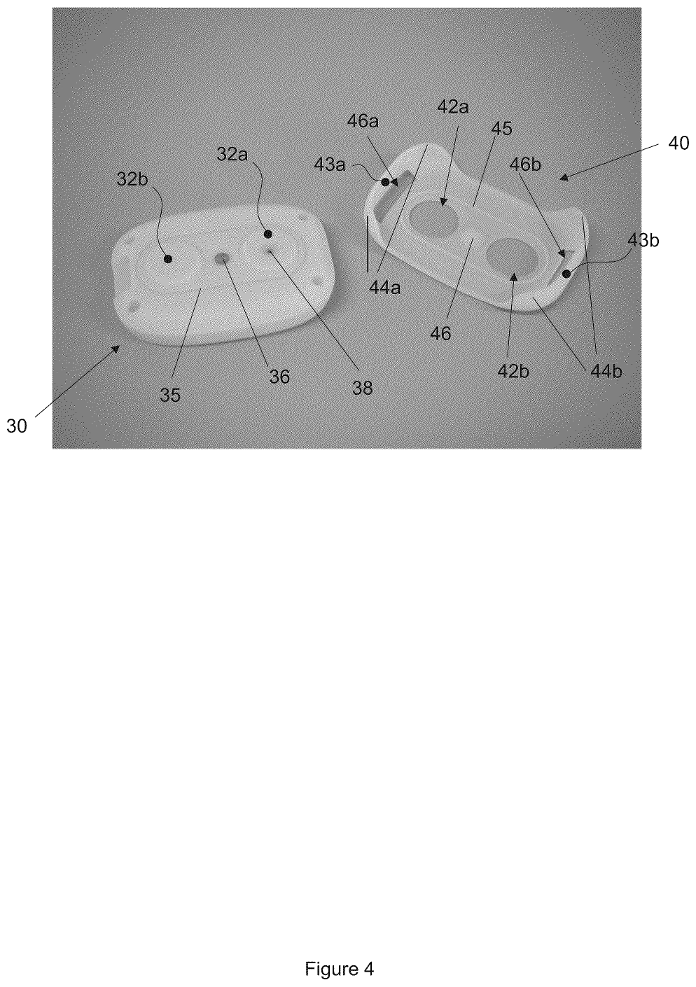

In an embodiment, the sound sensor 11 comprises an accelerometer of sufficient bandwidth located within the housing 30, suitably configured (e.g. by a suitably set gain parameter) to make measurements of the patients breathing, accounting for both the expected amplitude and expected frequency range(s) of such a signal. An advantage of this embodiment may be that the sound aperture 38 described with reference to FIG. 4 can be omitted (as the accelerometer can detect the vibrations associated with the patient's breathing without said sound aperture 38), thereby enabling the housing 30 to be sealed such as to prevent contamination between the internal components within the housing 30 and the patient's body.

In an embodiment, the movement sensor 12 comprises an accelerometer, such as a 3-axis accelerometer, and is configured to provide either an acceleration magnitude (i.e. independent of direction but calculated according to the measured acceleration in 3-dimensions) or an acceleration vector (i.e. the movement data comprises vector data). The movement sensor 12 is configured (e.g. by a suitably set gain parameter) to produce measurement of the expected movement of the patient (e.g. in particular, the patient's thorax) during a coughing event. This movement is typically very large compared to the amplitude of the sound of the coughing event, for example by one or more order of magnitudes, and of a relatively low frequency (e.g. below 10 Hz, such as of the order of 1 Hz).

In an embodiment, the sound sensor 11 and the movement sensor 12 utilise a common accelerometer. As noted, generally, there is an expectation that the movement of the patient (e.g., the patient's thorax) is much larger than the movement caused by the patient's breathing, including cough and wheeze sounds, but with much lower frequency. The accelerometer should have sufficient dynamic range to enable signal processing to isolate the relatively large amplitude but low frequency signal of the patient's movement during coughing with the relatively low amplitude but higher frequency sound vibrations. This embodiment advantageously may avoid a requirement for a sound aperture 38, thereby enabling the housing 30 to be sealed. For example, the accelerometer (either when used for the movement sensor 12 only or when used as a common accelerometer for the sound sensor 11 and the movement sensor 12) can be a Kionix® Tri-Axis Accelerometer (part number KX132-1211).

FIG. 1C shows an embodiment wherein the system 10 further comprises an auxiliary microphone 15 interfaced with controller 13. FIG. 1C specifically shows a variation to the embodiment of FIG. 1A, although it is understood to be applicable to the embodiment of FIG. 1B. The auxiliary microphone 15 is configured, in-use, to primarily make background sound measurements (“background measurements”) as opposed to making patient sound measurements. FIG. 1C also shows an optional temperature sensor 16 interfaced with the controller 13 which, in-use, measures the body temperature of the patient.

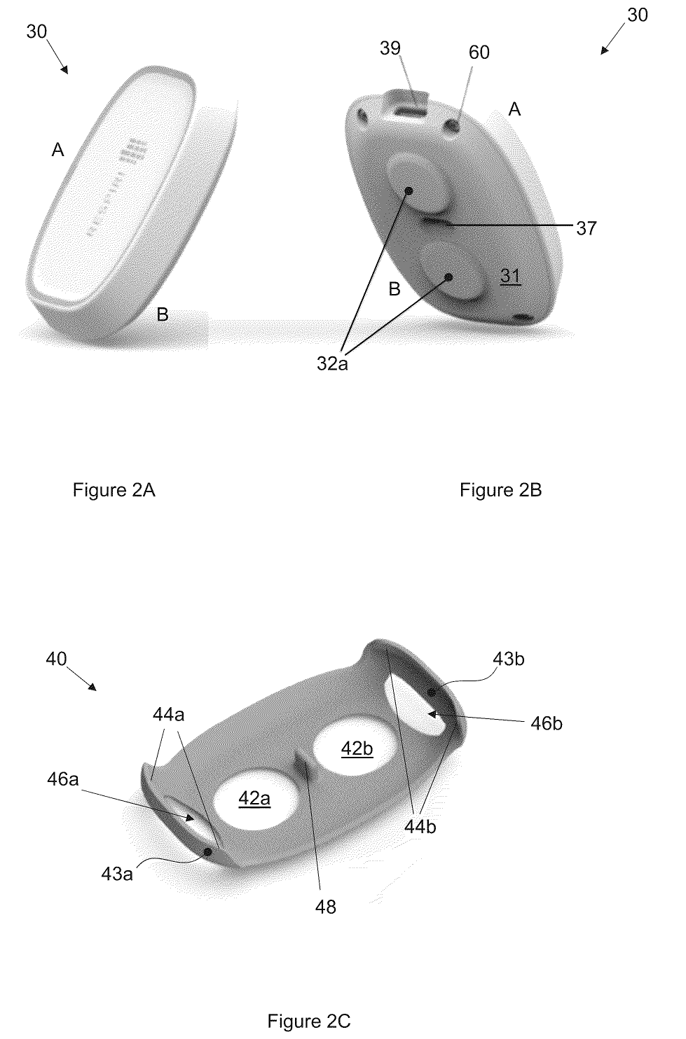

FIGS. 2A-2C show an embodiment in which the housing 30 is shaped for removeable attachment to a complementary holder 40, comprising two faces A and B. FIG. 2A shows an outward facing face A, which in use is not in contact with the patient.

FIG. 2B shows an inward facing face B (i.e. opposite to face A), which in use is (at least partially) in contact with the patient. The housing 30 comprises an interface portion 31 (shaded), which in the illustrated example includes substantially an entire face B and at least portions of the sides of the housing 30. The interface portion 31 comprises first and second protrusions 32a and 32b, which in the present case are substantial circular. More generally, the first protrusion 32a can be differently shaped to the second protrusion 32b. The first and second protrusions 32a, 32b correspond to active sensing portions of the housing 30, discussed in more detail below. In an embodiment, either or both of the first and second protrusions 32a, 32b defines an active sensing portion of the housing 30 but do not protrude from the surface B. FIG. 2B also shows an optional USB port 39 and screw bosses 60.

FIG. 2C shows the complementary holder 40. The figure shows an interacting side C of the holder 40 which, in use, abuts the inward facing face B of the housing 30. The holder 40 comprises, for each protrusion 32 of the housing 30, a complementary aperture 42 (i.e. 42a and 42b in FIG. 2C). Therefore, in use, the housing 30 is placed within the holder 40 such that each protrusion 32a, 32b extends through a respective aperture 42a, 42b.

In the embodiments shown, the protrusions 32a, 32b have the same cross-section as each other, and therefore, the apertures 42a, 42b are identical to one another. Additionally, the protrusions 32a, 32b are located symmetrically with respect to line A-A and the apertures 42a, 42b are located symmetrically with respect to line B-B. Advantageously, the shown embodiments may allow for ease of use by allowing the housing 30 to be attached to the holder 40 in either of two orientations. In another embodiment, not shown, the protrusions 32a, 32b are either asymmetrically arranged on the interface portion 31, of differing cross-sections, or both, and the apertures 42a, 42b are correspondingly arranged asymmetrically, have different cross-sections, or both. In this embodiment, an advantage may be provided where the housing 30 is required to be affixed to a patient with a particular orientation.

Referring still to FIG. 2C, the holder 40 is provided with attachment means for enabling attachment of the housing 40 to the housing 30. In the example shown, the attachment means comprise oppositely positioned clip portions 43a, 43b, each having a lip 44a, 44b. Generally, the attachment means allows for the housing 30 to be secured to the holder 40 in-use, while allowing for ease of detachment (e.g. by allowing a relatively modest force to effect detachment, preferably no stronger than that which can be provided by a typical adult user, preferably with one hand).

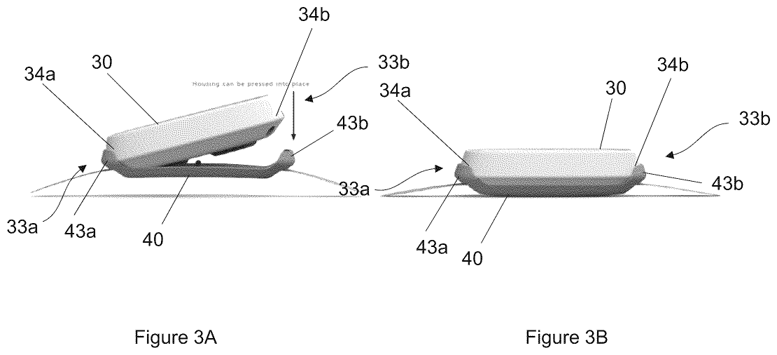

FIGS. 3A and 3B illustrate the interaction of the attachment means to enable the housing 30 to be secured to the holder 40. Both figures show a side profile of the housing 30 and the holder 40.

In FIG. 3A, the housing 30 has been partially brought into contact with the holder 40, but not yet secured. As can be seen, a first end 33a of the housing 30 has been placed into contact with the holder 40. Attachment is achieved by the user pushing the housing 30 onto the holder 40, thereby bringing the other second end 33b into contact with the holder 40, as shown in FIG. 3B. The clip portions 43a, 43b and lips 44a, 44b (hidden in FIGS. 3A and 3B) interact with complementary features on the housing 30 in order to secure the housing 30 to the holder 40. In the embodiment shown, ends 33a, 33b of the housing taper from a widest point towards the outward facing face A, thereby providing respective shoulders 34a, 34b at each widest point. With reference to FIG. 2C, the lips 44a, 44b are shaped to overlap the shoulders 34a, 34b thereby securing the housing 30 to the holder 40.

The holder 40 is formed, at least in part, from a resilient material to enable sufficient movement of the clip portions 43 that the housing 30 can be inserted into the in-use configuration. In the embodiment shown, the holder 40 is formed from a unitary resilient material, although in alternatives the holder 40 includes resilient material and another material, which may be rigid.

FIG. 4 shows additional locating means of the housing 30 and holder 40, according to an embodiment. In a first example, the interface portion 31 of the housing 30 includes a narrow recess 35, in this example surrounding the first and second protrusions 32a, 32b. The holder 40 comprises a complementary narrow ridge 45 surrounding the first and second apertures 42a, 42b. In use, with the housing 30 affixed to the holder 40, the ridge 45 is located within the recess 35, thereby providing locating means for the housing 30 with respect to the holder 40. In a second example, the interface portion 31 of the housing 30 includes a circular indent or aperture 36, in this example positioned equidistant between the first and second protrusions 32a, 32b. The holder 40 comprises a complementary protrusion 46 located between the first and second apertures 42a, 42b. In use, with the housing 30 affixed to the holder 40, the protrusion 46 is located within the indent or aperture 36, thereby providing another locating means for the housing 30 with respect to the holder 40. Generally, the additional locating means improves consistency and accuracy of an in-use relative positioning of the housing 30 and the holder 40.

The first and second protrusions 32a, 32b provide a contact point between the patient's body and the housing 30, as each extends through the holder 40 when in-use. The first protrusion 32a is associated with the sound sensor 11, allowing for vibrations generated by the patient to couple to the sound sensor 11 for detection, where the vibrations correspond to a detectable sound. Similarly, the second protrusion 32b is associated with the temperature sensor 16 and allow a physical coupling between the temperature sensor 16 and the patient's body to facilitate temperature measurement. Referring to FIG. 4, the housing can be provided with a sound aperture 38 to facilitate vibrational coupling between the sound sensor 11 and the patient's body.

In use, the holder 40 and housing 30 are affixed the patient such as to provide contact between the first and second protrusions 32a, 32b and the patient. For example, the holder 40 and housing 30 can be located at the patient's thorax. Typically, the first and second protrusions 32a, 32 are required to be in contact directly with the body (i.e. skin) of the patient.

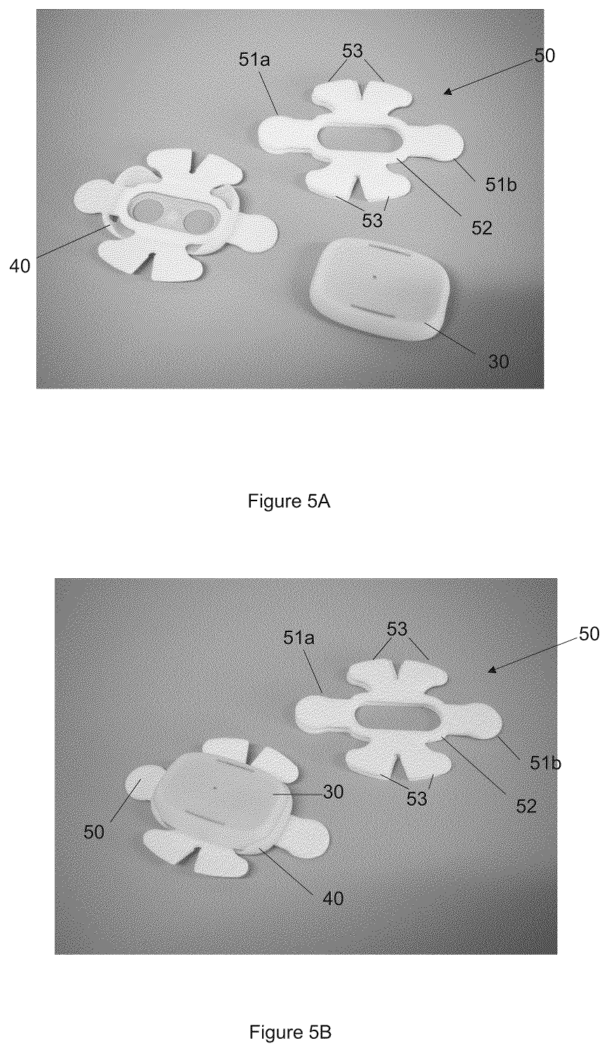

FIGS. 5A and 5B show securing means for securing the holder 40 to the body of the patient, and thereby securing the housing 30 to the patient. In the embodiment shown, the securing means comprises adhesive element 50 having a geometry, in a plan-view (as shown), configured to enable the adhesive element 50 to interface with the holder 40 while providing contact portions between the adhesive element 50 and the patient's body. In the example, the adhesive element 50 comprises a central portion 52 with a first securing tab 51a and a second securing tab 51b extending from the central portion 52 in opposite directions from one another. The adhesive element 50 also includes additional tabs 53 which extend in roughly perpendicular directions with respect to an axis extending between the two securing tabs 51a, 51b.

In-use, the securing tabs 51a, 51b are thread through respective securing apertures 49a, 49b within the clip portions 43a, 43b of the holder 40. Therefore, the central portion 52 is substantially located, in used, between the holder 40 and the housing 30. As shown, central portion 52 does not cover apertures 42a, 42b of the holder 40, thereby avoiding interfering with protrusions 32a, 32b of the housing 30.

The geometric configuration of the adhesive element 50 allows it to be held within the holder 40 in a correction position, while retaining a relative ease of use. The securing tabs 51 and, where applicable, additional tabs 52 have an adhesive present on their body-facing surfaces (i.e. the surfaces facing away from the housing 30 in-use). The adhesive should be selected for safe but secure attachment of the adhesive element 50 to the body of the patient (which will typically include contact with the skin of the patient). Such adhesives are well-known in the art.

Advantage of providing a separate adhesive element 50 to the holder 40 may be that it enables single-use of the adhesive element 50 and multi-use of the holder 40, with respect to different patients. Thus, the same adhesive is not used on multiple patients. Another advantage may be that the adhesive element 50 is relatively cheaper to produce that the holder 40, thereby reducing overall costs.

Referring back to FIG. 2b, according to an embodiment, a concealed power switch 37 is provided within a recess of the interface portion 31. The power switch 37 can be actuated by a user directly (e.g. by pressing or sliding the switch, depending on the implementation). However, referring back to FIG. 2C, in an embodiment, the holder 40 comprises a complementary power activator 48 corresponding to a protrusion which, when in-use, activated the power switch 37 via a pressing action. In this way, the controller 13 and interfaced components are put into an active mode through the action of attaching the housing 30 to a holder 40. It should be understood that, in an embodiment, the controller 13 and optionally one or more other components can remain powered when the power switch 37 is not actuated; however, the controller 13 is in this case placed into in an inactive mode, which typically corresponds to a low power mode when compared to the active mode. In an embodiment, the controller 13 can be put into an active mode via a data communication received via the communication module 14.

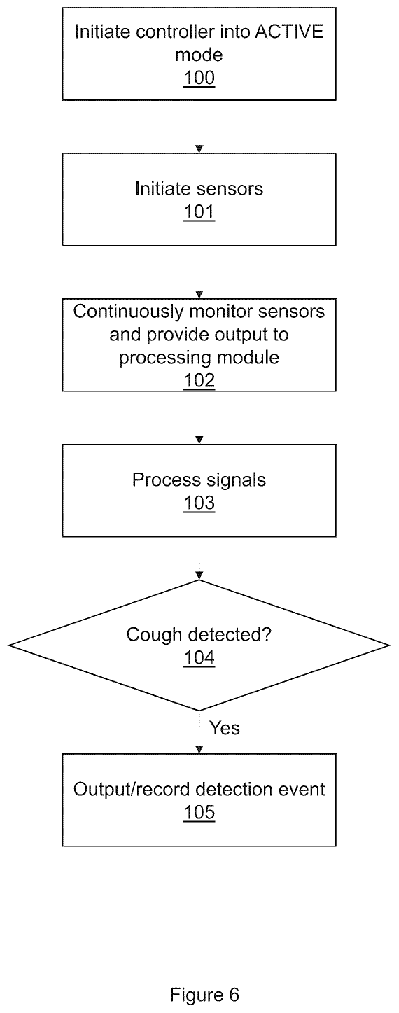

FIG. 6 shows a method for monitoring the breathing of a patient using the cough detection system 10. First, at step 100, the controller 13 enters an active mode of operation. The controller 13 then initiates the various interfaced sensors, in particular, the sound sensor 11 and movement sensor 12, at step 101.

At step 102, the controller 13 continuously receives sound data from the sound sensor 11 and produces a digitised breath signal. The sound data can be analogue data which is digitised by the controller 13 or the sound sensor 11 can be associated with digitising electronics configured to convert the analogue raw sound signal into a digitised sound signal. In either case, the digital breath signal is provided to the processing module 20.

Also, at step 102, the controller 13 continuously receives movement data from the movement sensor 12 and produces a digitised movement signal. For example, where the movement sensor 12 comprises an accelerometer, the movement data comprise acceleration data. The movement data can be analogue data which is digitised by the controller 13 or the movement sensor 12 can be associated with digitising electronics configured to convert the analogue raw movement signal into a digitised movement signal. In either case, the digital movement signal is provided to the processing module 20.

Similarly, depending on the embodiment, the controller 13 receives additional data from one or more additional sensors, which can include one or both of the auxiliary microphone 15 and temperature sensor 16. As with the sound sensor 11 and the movement sensor 12, the controller 13 is configured to produce to the processing module 20 digital signals associated with each additional sensor.

As previously described, in an embodiment, the controller 13 implements the processing module 20 itself, in which case, the various digital signals are provided to the processing module 20 via a shared memory. In embodiments where the processing module 20 is implemented by the processing system 19, the controller 13 is configured to communicate, using a suitable wired or wireless protocol, the various digital signals to the processing module 20 via the communication module 14. It should be understood, therefore, that the various signals are provided via storage of data values of the signals in memory.

At step 103, the processing module 20 is configured to undertake signal processing operations in respect of the sound signal, the movement signal, and where applicable, any additional signal(s).

According to an embodiment, the processing operations comprise identification of a coincidence with respect to identification of a predefined sound pattern within the sound signal and identification of a predefined movement pattern within the movement signal. Here, coincidence refers to the predefined sound pattern and the predefined movement pattern occurring at the “same time”, although it should be understood that coincidence is typically associated with a time window—that is, the identified predefined sound pattern occurs within a window of the predefined movement pattern, and/or vice versa.

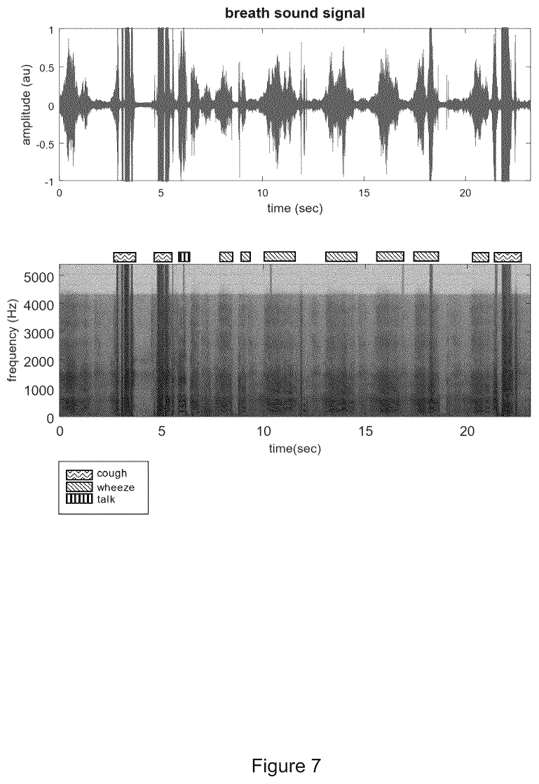

FIG. 7 shows a breath signal as measured of a patient suffering from impaired respiratory capability. The patient experienced periods of coughing and periods of wheezing, and also spoke (see key). The top plot shows a measured sound signal (showing amplitude over time) and the bottom plot shows a calculated frequency spectrum of the sound over time. The plots are aligned to show respective amplitude and frequency distributions at each time segment.

The Applicant's existing device, Wheezo™, is configured to detect wheezing from short audio recordings. Sound recordings of a patient's breathing are analysed for the spectral features that distinguish wheeze from normal breathing sounds. Wheeze is distinguishable from normal breathing by the presence of clear pitch due to resonance of partially restricted airways in the lungs. A feature observed from Wheezo recordings is that the amplitude of the sound varies little between normal breathing and wheeze. Reference is made to Applicant's Australian provisional application no. 2020903832, filed on 22 Oct. 2020, which describes a method for identifying wheeze in contrast to regular breathing sounds, the entire content of which is incorporated herein by reference. PCT application no. PCT/AU2021/051232, filing date 22 Oct. 2021 and published as WO 2022/082272 A1 on 28 Apr. 2022, which claims convention priority to Australian provisional application no. 2020903832, is also incorporated herein by reference in its entirety.

In contrast, a cough is a respiratory manoeuvre that is a reflexive response to clear the airways. A cough is a quick sequence inspiration to store air, closing of the glottis with activation of expiratory muscles to build pressure, and finally an explosive release to clear the upper airways. In an audio recording, cough is evident as large amplitude and short duration bursts with a broad spectral pattern. FIG. 7 shows real-world examples of the amplitude and frequency spectra of both coughing and wheezing.

However, merely processing the audio recording for large amplitude, broad spectrum bursts is expected to result in a reasonably high number of false positives; that is, such an approach is likely to detect as coughs extraneous signals having similar sound characteristics.

Therefore, the processing module 20 is configured to identify both a predefined sound pattern corresponding to a cough and a predefined movement pattern corresponding to thoracic motion. As the housing 30 is placed on the thorax of the patient, and sudden movement coincident with a detected cough in the sound signal can be considered evidence that a cough has actually occurred, thereby advantageously reducing or eliminating instances of false positive detections.

The predefined sound pattern can be parameterised—that is, the user can configure the processing module 20 by modifying one or more modifiable parameters. This can enable the user to set the processing module 20 as desired, for example, for greater sensitivity with a greater risk of false positives or a lower sensitivity with a lower risk. Similarly, the predefined movement pattern can be parameterised for similar reasons.

It should be clear that both the predefined sound pattern and the predefined movement pattern effectively define a class of patterns, and therefore, each effectively defines a range of different specific signals.

If the processing module 20 identifies a cough detection event (see step 104), then it generates a detection alert at step 105. However, as detection is continuous, the method continues monitoring (i.e. step 102 is perpetual while in the active mode).

The detection alert can be output to a memory storage of the processing system 19—that is, output should be understood as encompassing, where applicable, storing in memory. The detection alert can also correspond to a message being communicated to another computer system or a local indicator (e.g. a visual and/or audible output).

Depending on the embodiment, the processing module 20 can undertake analysis contemporaneously with signal generation (i.e. in effective “real-time”) or the processing module 20 can undertake post-processing of the data. For example, the generation of a detection alert can be performed in response to a user issued command to the processing module 20 at a time subsequent to actual monitoring of the patient.

Generally, embodiments described herein provide an accurate means for determining coughing events of a patient. This information can be analysed in real-time to enable at the time intervention; for example, a large number of coughs within a time period may alert a health professional to attend to a patient. The information can also be analysed for post-event analysis.

Further modifications can be made without departing from the spirit and scope of the specification.

1. A wearable apparatus for a patient for use in cough detection, comprising:
a movement sensor configured to generate measurements of a movement of the patient, the movement sensor configured to be worn by the patient;
a sound sensor configured to generate measurements of breathing sounds of the patient;
a controller interfaced with the movement sensor and the sound sensor; and
a housing for locating the movement sensor, sound sensor, and controller,
wherein the controller is configured to:
obtain a movement signal corresponding to the measurements of the movement of the patient made by the movement sensor;
obtain a sound signal corresponding to the measurements of the breathing sounds of the patient made by the sounds sensor;
generate thoracic movement data from the movement signal;
generate breathing data from the sound signal; and
generate respiratory data comprising the thoracic movement data and the breathing data, and further comprising correlation information configured to enable identification of contemporaneous breathing events within the thoracic movement data and the breathing data;
provide correlated respiratory data to a processing module configured for identifying specific breathing events at least including coughing events,
such that a coughing event is identifiable from the correlated respiratory data according to a coincidence of identification of a predefined sound pattern within the breathing data and identification of a predefined movement pattern within the thoracic movement data.

2. The wearable apparatus of claim 1, wherein the housing comprises an external surface defining an interface portion substantially comprising a first face of the body, wherein, in-use, the interface portion is configured to be proximal a surface of the patient.

3. The wearable apparatus of claim 2, wherein the interface portion comprises at least one protrusion configured for contact with the patient, the, or each, protrusion associated with a sensor of the wearable apparatus.

4. The wearable apparatus of claim 1, further comprising a holder, the holder having a shape complementary to a profile of at least the interface portion of the housing such that the housing is attachable to and detachable from the holder, wherein the holder is configured to securely hold the housing during use.

5. The wearable apparatus of claim 4, wherein the housing comprises an external surface defining an interface portion substantially comprising a first face of the body, wherein, in-use, the interface portion is configured to be proximal a surface of the patient, wherein the interface portion comprises at least one protrusion configured for contact with the patient, the, or each, protrusion associated with a sensor of the wearable apparatus and wherein an aperture is provided in the holder for the, or each, protrusion of the interface portion, such that the, or each, protrusion extends through its associated aperture thereby enabling contact between the protrusion and a surface of the patient.

6. The wearable apparatus of claim 4, wherein the holder comprises one or more clip portions configured to interact with associated complementarily shaped one or more end portions of the housing for enabling secure attachment of the holder and the housing.

7. The wearable apparatus of claim 4, wherein the housing includes a recess comprising an operational switch and the holder comprises a complementary power activating being a protrusion configured to actuate the operational switch when the housing is securely held by the holder.

8. The wearable apparatus of claim 4, further comprising an adhesive element having a geometry such that, when coupled to the holder, one or more tabs extend away from the holder, said one or more tabs including an adhesive layer for securing the, or each, tab to the patient thereby enabling the holder to be secured to the patient.

9. The wearable apparatus of claim 1, further comprising an auxiliary microphone arranged to substantially measure background sound to enable removal of said measured background sound from the measurements of the breathing sounds of the patient.

10. The wearable apparatus of claim 1, further comprising a temperature sensor configured to produce temperature measurements of a temperature of the patient, and wherein the controller is further configured to:
obtain a temperature signal corresponding to the measurements of the temperature of the patient made by the temperature sensor;
generate temperature data from the temperature signal; and
include the temperature data with the respiratory data
wherein the correlation information is further configured to enable identification of contemporaneous temperatures of the patient associated with specific breathing events.

11. The wearable apparatus of claim 1, wherein:
the sound sensor comprises a microphone such as a MEMS microphone; and/or
wherein the movement sensor comprises an accelerometer.

12. The wearable apparatus of claim 1, wherein the sound sensor comprises an accelerometer.

13. The wearable apparatus of claim 12, wherein the movement sensor utilises the accelerometer of the sound sensor.

14. A cough detection system for the detection of coughs by a patient, the system comprising;
the wearable apparatus of claim 1; and
the processing module,
wherein the processing module is configured to:
analyse the breathing data to identify instances of the breathing signal consistent with a predefined characteristic cough pattern,
analyse the thoracic movement data to determine instances of a thoracic movement corresponding to a predefined characteristic movement pattern,
in response to identifying a correlation, in time, of an instance of a predefined cough pattern and an instance of a predefined movement pattern, assign the instances as corresponding to a cough.

15. The cough detection system of claim 14, wherein the predefined characteristic cough pattern is characterised by a relatively large amplitude compared to non-cough breathing sounds having short duration bursts with a broad spectral pattern.

16. The cough detection system of claim 14, wherein the predefined characteristic movement pattern is characterised by a relatively large movement of the patient's thorax compared to the magnitude of the amplitude of the signal generated by the sound sensor.

17. The cough detection system of claim 16, wherein the predefined characteristic movement pattern is at least in part defined by one or more settable parameters, such that a user is enabled to configure the predefined characteristic movement pattern.

18. The cough detection system of claim 14, wherein the processing module is implemented by the controller of the wearable apparatus.

19. The cough detection system of claim 14, further comprising a processing sever separate to the wearable apparatus, and wherein the processing module is implemented by the processing server, and wherein the wearable apparatus is configured to communicate the respiratory data to the processing server via one or more data communication protocols.

20. A method for processing respiratory data, wherein the respiratory data is obtained, at least in part, by a wearable apparatus comprising a sound sensor and a movement sensor, comprising the steps of:
obtaining respiratory data comprising thoracic movement data corresponding to measurements of a movement of the patient's thorax made by the movement sensor and the breathing data corresponding to measurements of breathing sounds of the patient made by the sound sensor, and further comprising correlation information configured to enable identification of contemporaneous breathing events within the thoracic movement data and the breathing data;
analysing the breathing data to identify instances of the breathing signal consistent with a predefined characteristic cough pattern,
analysing the thoracic movement data to determine instances of a thoracic movement corresponding to a predefined characteristic movement pattern, and
in response to identifying a correlation, in time, of an instance of a predefined cough pattern and an instance of a predefined movement pattern, assigning the instances as corresponding to a cough.

21. The method of claim 20, further comprising the steps of:
obtaining, by a controller of the wearable apparatus, a movement signal corresponding to the measurements of the movement of the patient made by the movement sensor;
obtaining, by a controller of the wearable apparatus, a sound signal corresponding to the measurements of the breathing sounds of the patient made by the sounds sensor;
generating, by a controller of the wearable apparatus, thoracic movement data from the movement signal;
generating, by a controller of the wearable apparatus, breathing data from the sound signal; and
generating, by a controller of the wearable apparatus, the respiratory data.
